(19) United States
(12) Patent Application Publication
(10) Pub. No.: US 2015/0099751 A1
(43) Pub. Date: Apr. 9, 2015

(54) IN SITU GEL LOADED WITH PHOSPHODIESTERASE TYPE V INHIBITORS NANOEMULSION

(71) Applicant: KING ABDULAZIZ UNIVERSITY, Jeddah (SA)

(72) Inventors: HISHAM AHMED MOHAMMED MOSLI, JEDDAH (SA); SALAH ABDELMONEIM GHAREIB ATTEIAH, JEDDAH (SA); KHALED MOHAMED HOSNY OMAR, JEDDAH (SA); HANY MOHAMED ABD EL-MALIK EL-BASSOSSY, JEDDAH (SA); MOHAMED HANY ABDULWAHAB MOSILHY HELAL, JEDDAH (SA)

(73) Assignee: KING ABDULAZIZ UNIVERSITY, Jeddah (SA)

(21) Appl. No.: 14/047,928

(22) Filed: Oct. 7, 2013

(51) Int. Cl. A61K 9/107; A61K 31/53; A61K 31/4985; A61K 31/506; A61K 47/34; A61K 47/44; A61K 47/36; A61K 9/00; A61K 47/10; A61K 31/519; A61K 9/06

(52) U.S. Cl. CPC A61K 9/1075; A61K 31/519; A61K 31/53; A61K 31/4985; A61K 31/506; A61K 9/06; A61K 47/44; A61K 47/36; A61K 9/0019; A61K 47/10; A61K 47/34
USPC 514/243; 514/252.16; 514/250; 514/262.1; 514/261.1; 514/252.11; 514/275

(57) ABSTRACT

The in situ gel loaded with phosphodiesterase Type V (PDE5) inhibitors nanoemulsion was prepared using an innovative approach to reformulate the PDE5 inhibitors in a nanoemulsion, and then loading the nanoemulsion into an in situ gel base. These preparations are administered by intramuscular injection in order to give a depot effect for a period of time that exceeds 15 days. The in situ gel composition gives slow, controlled release of the PDE5 inhibitor. The in situ gel composition is useful for prophylaxis and treatment of some important chronic diseases, such as diabetic complications, benign prostatic hyperplasia, erectile dysfunction, and diseases associated with endothelial dysfunction.

### BACKGROUND OF THE INVENTION

1. Field of the Invention

The present invention relates to pharmaceutical delivery agents and carriers, and particularly to an in situ gel loaded with phosphodiesterase Type V inhibitors nanoemulsion for delivery by intramuscular injection.

2. Description of the Related Art

Phosphodiesterase Type V inhibitors (PDE5 inhibitors) are available in the market as oral tablets. These tablets have a lot of side effects, such as (1) gastrointestinal side effects resulting in dyspepsia, burning sensation; (2) slow onset of action, which is attained after 60 minutes; (3) extensive first pass metabolism; (4) low bioavailability due to low solubility and extensive metabolism; (5) extensive food-drug interaction, especially with fatty foods, which hinder its absorption; and (6) most of them require frequent dosing due to their short half-life.

An example of a PDE5 inhibitor drug is Sildenafil, 1[4ethoxy3(6.7dihydro1methyl7oxo3propyl1Hpyrazolo [4,3d] pyrimidin5yl) phenylsulfonyl]4methylpiperazine, sold under the brand name Viagra® (Viagra is a registered trademark of Pfizer Inc. of New York, N.Y.). Sildenafil is the most common publically available drug indicated in the treatment of erectile dysfunction. It was primarily meant for the treatment of angina and hypertension, but was found to have a strong positive effect on sexual performance. It was approved by the FDA (Food and Drug Administration) in March 1998. The recommended sildenafil dose is 50 mg approximately one hour before sexual activity.

Sildenafil is absorbed following administration by mouth (orally), and has low bioavailability of approximately 40% and an onset of action that is attained within 60 minutes. The drug has a duration of action lasting up to 4 hours, but with less response than that seen at 2 hours. Oral sildenafil is effective for treatment of erectile dysfunction of various etiologies. At the recommended doses, sildenafil has no direct relaxant effect on the isolated human corpus cavernosum, but it enhances the effect of NO (nitric oxide) by inhibiting phosphodiesterase5 (PDE5); the enzyme which is responsible for the breakdown of cGMP in the corpus cavernosum, and thus reported to sustain the penile erection. Without sexual stimulation, sildenafil has no effect on erection.

There is evidence from preliminary clinical studies that NO donor drugs and PDE5 inhibitors, such as sildenafil, tadalafil (Cialis®, a registered trademark of Lilly ICOS LLC of Wilmington, Del.) and vardenafil (Levitra®, a registered trademark of Bayer Aktienggesellschaft of Germany), which are assumed to act by relaxing prostate or/and bladder smooth muscle, can improve male lower urinary tract syndrome (LUTS). After 12 weeks of treatment with sildenafil, there was an overall improvement in the International Prostate Symptom Score (IPSS) and LUTS-specific quality of life (QoL) score. In addition, vardenafil might offer a valuable new option for the treatment of LUTS associated with benign prostatic hyperplasia (BPH).

It was reported that the NO donor drugs and PDE5 inhibitors effect on male LUTS might be explained by their potential relaxant effect on the smooth muscle of the prostate, urethra and bladder/bladder neck, as well as on pelvic vessels supplying the prostate and bladder. Based on preliminary data derived from in vitro studies on human and animal tissues, the chronic administration of NO donor drugs and PDE5 inhibitors may also induce anti-proliferative and/or apoptotic effects in the prostate, thus corresponding with clinical efficacy in terms of progression of LUTS.

Moreover, the PDE5 inhibitors are used nowadays in treatment of Pulmonary Arterial Hypertension (PAH) in children. Revatio® (sildenafil citrate) (TM Pfizer Product Inc. ©Pfizer Canada Inc., 2012) is indicated for treatment of primary pulmonary arterial hypertension (PPH) or pulmonary hypertension secondary to connective tissue disease in patients with WHO functional class II or III who have not responded to conventional therapy. The recommended dose of Revatio® for oral administration is 20 mg three times a day (t.i.d.). However, the recommended dose of Revatio® for intravenous administration is 10 mg (corresponding to 12.5 ml) three times a day, administered as an intravenous bolus injection.

The major adverse effects of sildenafil are gastrointestinal effects resulting in dyspepsia and burning sensation. Moreover, absorption of sildenafil is hindered by fatty meals, with a mean delay in Tmax of 60 minutes and a mean reduction in Cmax of 29%.

Thus, an in situ gel loaded with phosphodiesterase Type V inhibitors nanoemulsion solving the aforementioned problems is desired.

### SUMMARY OF THE INVENTION

The in situ gel loaded with phosphodiesterase Type V inhibitors nanoemulsion was prepared using an innovative approach to reformulate the PDE5 inhibitors in a nanoemulsion, and then loading the nanoemulsion into an in situ gel base. These preparations are administered by intramuscular injection in order to give a depot effect for a period of time that exceeds 15 days.

These and other features of the present invention will become readily apparent upon further review of the following specification.

### DETAILED DESCRIPTION OF THE PREFERRED EMBODIMENTS

The in situ gel composition loaded with phosphodiesterase Type V inhibitors nanoemulsion gives slow, controlled release of the inhibitor(s) and is administered as an intramuscular injection. The in situ gel composition is useful for prophylaxis and treatment of some important chronic diseases, such as diabetic complications, benign prostatic hyperplasia, erectile dysfunction, and diseases associated with endothelial dysfunction.

The in situ gel composition loaded with phosphodiesterase Type V inhibitors nanoemulsion was prepared using an innovative approach to reformulate PDE5 inhibitors in a nanoemulsion, and then the nanoemulsion was loaded into an in situ gel base. The PDE5 inhibitors used in the preparation of the in situ gel composition of present invention may include any of the following phosphodiesterase5 inhibitors: Sildenafil, Verdinafil, Tadalafil, Udenafil, Zaprinast, Lodenafil, Mirodenafil, Sulfoaildenafil, Avanafil, or Aildenafil, either individually or in combination, in individual amounts ranging from 20 milligrams to 300 milligrams. The in situ gel composition is administered as an intramuscular injection in order to give a depot effect for period of time that exceeds 15 days.

The slow release character of the depot preparation will provide a stable concentration of the PDE5 inhibitor in the blood for a long time. Use of the in situ gel composition results in increased patient compliance with the medication, improvement in the PDE5 inhibitors' bioavailability, and reduction or even removal of the known side effects of the commercially available tablets of PDE5 inhibitors.

The relatively low blood concentration of PDE5 inhibitors delivered by this medical preparation over a long period of time will be beneficial for prophylaxis and treatment of some important chronic diseases, such as diabetic complications, benign prostatic hyperplasia, erectile dysfunction, and diseases associated with endothelial dysfunction.

The diabetic complications include cardiovascular complications; cardiomyopathy, microangiopathy, retinopathy, nephropathy, macroangiopathy and atherosclerosis. Cardiomyopathy is a measurable deterioration of the function of the myocardium (the heart muscle) that can result from untreated or poorly treated long-term diabetes. Microangiopathy is an angiopathy (i.e., disease of the blood vessels) affecting small blood vessels in the body that can result from untreated or poorly treated long-term diabetes. Retinopathy is damage to the retina of the eye that can result from untreated or poorly treated long-term diabetes. Nephropathy is damage to or disease of a kidney that can result from untreated or poorly treated long-term diabetes. Macroangiopathy is an angiopathy of the greater blood vessel in the body that can result from untreated or poorly treated long-term diabetes. Atherosclerosis is an arteriosclerotic vascular disease is a condition in which an artery wall thickens as a result of the accumulation of fatty materials, such as cholesterol. It is a syndrome affecting arterial blood vessels that can result from untreated or poorly treated long-term diabetes.

Benign prostatic hyperplasia involves hyperplasia of prostatic stromal and epithelial cells, resulting in the formation of large, fairly discrete nodules in the periurethral region of the prostate. Erectile dysfunction is sexual dysfunction characterized by the inability to develop or maintain an erection of the penis during sexual performance. The diseases associated with endothelial dysfunction are such diseases as (but not limited to) hypertension and hypercholesterolaemia.

The nanoemulsion for the in situ gel composition was prepared using selected oils, surfactants and cosurfactants. The oils include arachis oil, jojoba oil, coconut oil, sesame oil, olive oil, castor oil, sunflower oil, Sefsol, Miglyol 812, Labrafil M1944, Labrafac, Triacetin, isopropyl myristate, oleic acid, linoleic acid, clove oil, and paraffin oil. The surfactants include Span® 20, Span® 80, Tween® 20, Tween 80, Cremophor EL and Labrasol. The cosurfactants include PEG 4000, PEG 6000, methanol, ethanol, isopropanol and propylene glycol.

To prepare the nanoemulsion for the nanoemulsionbased in situ gel composition, surfactant and cosurfactant (e.g., Smix) were mixed at different mass ratios (1:1, 2:1, 3:1, 4:1, and 5:1, 6:1, 1:2, 1:3, 1:4, 1:5, 1:6). These ratios were chosen in increasing concentration of surfactant with respect to cosurfactant. Oil and Smix at a specific ratio was mixed thoroughly at different mass ratios from 0.5:9.5 to 9.5:0.5 in different glass vials. Different combinations of oil and Smix were made so that maximum ratios were obtained. Then, the PDE5 inhibitors were loaded into the nanoemulsion.

The nanoemulsionbased in situ gel composition was prepared using gelling agents selected from the group consisting of Chitosan (low, medium, and high molecular weight), poly (d,l-lactidecoglycolide), Polycabrolactone, Poly(orthoesters), Gellan gum, Alginic Acid, Pluronic 127 and Carbomere. The in situ gelling agent constitutes from about 0.5 weight percent to about 10 weight percent based on the weight of the composition.

To prepare the nanoemulsion-based in situ gel composition, weighed amounts of the gelling agent were sprinkled gently in sufficient quantity of suitable solvent and stirred at high speed. Stirring was continued until a thin hazy dispersion without lumps was formed, and then the PDE5inhibitor loaded nanoemulsion was slowly added to the solution of gelling agent under continuous stirring.

It is to be understood that the present invention is not limited to the embodiments described above, but encompasses any and all embodiments within the scope of the following claims.

1. A composition for depot slow, controlled release of a PDE5 inhibitor, comprising a nanoemulsion containing an effective amount of at least one phosphodiesterase Type V (PDE5) inhibitor, the nanoemulsion loaded into an in situ gel base comprising an in situ gelling agent,
wherein the effective amount of the at least one PDE5 inhibitor comprises between 20 milligrams and 300 milligrams, and
the nanoemulsion comprises a blend of at least one oil, at least one surfactant, and at least one cosurfactant.

2. The composition of claim 1, wherein the at least one PDE5-inhibitor comprises at least one PDE5 inhibitor selected from the group consisting of Sildenafil, Verdinafil, Tadalafil, Udenafil, Zaprinast, Lodenafil, Mirodenafil, Sulfoaildenafil, Avanafil and Aildenafil.

3. (canceled)

4. (canceled)

5. The composition of claim 1, wherein the at least one oil comprises at least one oil selected from the group consisting of arachis oil, jojoba oil, coconut oil, sesame oil, olive oil, castor oil, sunflower oil, Sefsol, Miglyol 812, Labrafil M1944, Labrafac, Triacetin, isopropyl myristate, oleic acid, linoleic acid, clove oil, and paraffin oil.

6. The composition of claim 1, wherein the at least one surfactant comprises at least one surfactant selected from the group consisting of Span® 20, Span® 80, Tween® 20, Tween 80, Cremophor EL and Labrasol.

7. The composition of claim 1, wherein the at least one cosurfactant comprises at least one cosurfactant selected from the group consisting of PEG 4000, PEG 6000, methanol, ethanol, isopropanol and propylene glycol.

8. The composition of claim 1, wherein the molar ratio of the at least one surfactant to the at least one cosurfactant in the nanoemulsion is between 1:1 and 6:1 and between 1:1 and 1:6.

9. The composition of claim 1, wherein the blend of the at least one surfactant and the at least one cosurfactant comprises Smix and the ratio of the amount by weight of the at least one oil to the amount by weight of the Smix is between 0.5:9.5 and 9.5:0.5.

10. The composition of claim 1, wherein the in situ gelling agent is dispersed in sterile water.

11. The in situ gel composition of claim 10, wherein the in situ gelling agent comprises at least one gelling agent selected from the group consisting of Chitosan (low, medium, and high molecular weight), poly (d,l-lactidecoglycolide), Polycabrolactone, Poly(orthoesters), Gellan gum, Alginic Acid, Pluronic 127 and Carbomere.

12. The composition of claim 11, wherein the composition comprises between about 0.5% wt and about 10% wt of the in situ gelling agent.

13. A method of preparing a slow release formulation of a phosphodiesterase Type V (PDE5) inhibitor for intramuscular injection, comprising the steps of:
forming an emulsion from at least one oil, at least one surfactant, and at least one cosurfactant;
mixing an effective amount of a PDE5 inhibitor into the nanoemulsion;
mixing a gelling agent with a suitable solvent to form a gel carrier; and
mixing the PDE5 inhibitor-loaded emulsion into the gel carrier.

14. The method of preparing a slow release formulation according to claim 13, wherein the at least one oil comprises at least one oil selected from the group consisting of arachis oil, jojoba oil, coconut oil, sesame oil, olive oil, castor oil, sunflower oil, Sefsol, Miglyol 812, Labrafil M1944, Labrafac, Triacetin, isopropyl myristate, oleic acid, linoleic acid, clove oil, and paraffin oil.

15. The method of preparing a slow release formulation according to claim 14, wherein the at least one surfactant comprise at least one surfactant selected from the group consisting of Span® 20, Span® 80, Tween® 20, Tween 80, Cremophor EL and Labrasol.

16. The method of preparing a slow release formulation according to claim 15, wherein the at least one cosurfactant comprises at least one cosurfactant selected from the group consisting of Smix, PEG 4000, PEG 6000, methanol, ethanol, isopropanol and propylene glycol.

17. The method of preparing a slow release formulation according to claim 16, wherein the at least one PDE5-inhibitor comprises at least one PDE5 inhibitor selected from the group consisting of Sildenafil, Verdinafil, Tadalafil, Udenafil, Zaprinast, Lodenafil, Mirodenafil, Sulfoaildenafil, Avanafil and Aildenafil.

18. The method of preparing a slow release formulation according to claim 17, wherein the gelling agent comprises at least one gelling agent selected from the group consisting of Chitosan (low, medium, and high molecular weight), poly (d,llactidecoglycolide), Polycabrolactone, Poly(orthoesters), Gellan gum, Alginic Acid, Pluronic 127 and Carbomere.
